(19) United States
(12) Patent Application Publication
(10) Pub. No.: US 2026/0297634 A1
(43) Pub. Date: Oct. 1, 2026

(54) BIOTECHNOLOGICAL PRODUCTION OF ACETONE

(71) Applicant: Evonik Operations GmbH, Essen (DE)

(72) Inventors: Jan Benedikt METTERNICH, Recklinghausen (DE); Jörg-Joachim NITZ, Essen (DE); Simon BECK, Münster (DE); Stephan KOHLSTRUK, Gladbeck (DE); Wilfried BLÜMKE, Schöneck (DE); Peter KREIS, Dortmund (DE)

(73) Assignee: Evonik Operations GmbH, Essen (DE)

(21) Appl. No.: 19/482,837

(22) PCT Filed: May 2, 2024
(86) PCT No.: PCT/EP2024/062079

§ 371 Date: Nov. 10, 2025

(30) Foreign Application Priority Data
May 11, 2023 (EP) 23172840.3

(51) Int. Cl. C12P 7/30; C12M 1/00; C12M 1/107; C12N 1/20; C12N 9/04; C12N 9/10; C12N 9/88; C12N 15/74; C12R 1/145

(52) U.S. Cl. CPC C12P 7/30; C12M 21/04; C12M 43/04; C12N 1/20; C12N 9/0006; C12N 9/1029; C12N 9/13; C12N 9/88; C12N 15/74; C12R 2001/145; C12Y 101/01001; C12Y 203/01009; C12Y 208/03008; C12Y 401/01004

(57) ABSTRACT

The present invention relates to a method of producing acetone from a gas composition by microbial fermentation, the method comprising:
contacting directly at least one genetically modified homoacetogenic bacterium with the gas composition, the gas composition comprising at least CO, CO₂, H₂ and CH₄;
   wherein the gas composition is an off-gas from at least one steam methane reformer and the off gas from the steam methane reformer is directly brought into contact with the genetically modified homoacetogenic bacterium; and the
   homoacetogenic bacterium is genetically modified to produce acetone from the gas composition.

### FIELD OF THE INVENTION

The present invention relates to a biotechnological method of producing ketones from off or waste gases. In particular, the method involves directly contacting the off or waste gas to suitable bacteria for fermentation of the gas without any prior step of preparing the gas for the fermentation process. The off or waste gas is from a steam methane reformer and the off or waste gas therefrom can be directly introduced for microbial fermentation and production of acetone.

### BACKGROUND OF THE INVENTION

Catalytic processes may be used to convert gases consisting primarily of CO, CO₂ and hydrogen (H₂) into a variety of fuels and chemicals. Micro-organisms may also be used to convert these gases into useful fuels and chemicals. These biological processes, although generally slower than chemical reactions, have several advantages over catalytic processes, including higher specificity, higher yields, lower energy costs and greater resistance to poisoning. Especially the use of acetogenic bacteria on various carbon sources to produce ethanol, acetate and/or other alcohols is well known. General application of genetically modified organisms in the production of feedstock chemicals comprising CO is disclosed at least in EP2678432B1.

The utilization of syngas as a carbon source for gas fermentation is also demonstrated in U.S. Pat. No. 8,263,372B2. EP3050968B1 also discloses syngas-based production of various alcohols.

Furthermore, at least WO 2015/085015 A1, EP2181195 B1, WO2010/121849 A1, and Nature Biotechnology 2022, 40, 335-344 disclose the production of acetone and other ketones from different gases using genetically modified organisms. In U.S. Pat. No. 8,376,736B2, Lanzatech disclosed the use of blast furnace off-gases for the production of acetone. However, the use of the blast-furnace off-gas for fermentation was contingent on prior cooling and pre-treatment of the off-gas to remove particles, long-chain hydrocarbons, and tars from the gas stream. This not only increased the time required for production of acetone from off-gases but also made the process more complicated and expensive to design and produce in a large-scale.

Acetone is an industrial solvent and precursor for at least methyl methacrylate (MMA) and polymethyl methacrylate (PMMA), and isobutylene which have a multitude of functions in industry. Acetone is also a precursor for production of jet fuels (Anbarasan, Nature, 491: 235-239, 2012).

However, the currently available methods of producing acetone from off or waste gases are inefficient and have at least one additional step of treating the off gas before bringing the gas in contact with the bacteria for fermentation. The low productivity and low concentrations of end products also results in higher energy costs for product purification.

Accordingly, it is desirable to find other sustainable raw materials, as starting materials for acetone production via biotechnological means which result in the same or higher yields and also cause less damage to the environment. In particular, there is a need for a simple and efficient biotechnological production of acetone from sustainable raw material.

### DESCRIPTION OF THE INVENTION

The present invention attempts to solve the problems above by providing a method for the industrial scale production of feedstock chemicals, particularly acetone based on off-gas fermentation. The acetone may be used for application in downstream production processes. In particular, the method involves the step of contacting off-gases from a steam reformer with at least one bacterial cell that is capable of converting the off-gas to acetone. The new combination of steam reformer with a coupled gas fermenter according to any aspect of the present invention allows for application of steam reformer gases (specifically their off-gas streams) in the production of valuable chemicals, including alcohols, acids, aldehydes, and ketones with acetone as the targeted main product and the commonly observed by-products acetate and ethanol.

An advantage of the method according to any aspect of the present invention is the implementation of a new source of off-gas for the fermentation-based production of valuable feedstocks. Additionally, the improved gas mixture composition of off-gas from a steam reformer, particularly a steam methane reformer, allows for direct application in the fermentation process, particularly anerobic fermentation process, of the off-gas without the necessity of prior pre-treatment or scrubbing.

In contrast to many other off-gas streams, for example at least blast furnace off gas streams, the steam reformer off-gas stream according to any aspect of the present invention does not contain significant oxygen concentrations. This may be one reason why the steam reformer off-gas stream may be directly used as feed stream for anerobic gas fermentation processes. Another advantage of the method according to any aspect of the present invention is that pre-cooling of the gas-stream is not required prior to contact with the bacteria for fermentation as the gas temperature is approximately 40° C. at the steam reformer outlet from which the steam reformer off-gas is released. The use of steam reformer off-gas as a feed stream for fermentation thus makes the biotechnological production of acetone less complicated, cheaper and faster as the several steps of preparing the off-gas as a feed stream is not required.

According to one aspect of the present invention, there is provided a method of producing acetone from a gas composition by microbial fermentation, the method comprising:
contacting directly at least one genetically modified homoacetogenic bacterium with the gas composition, the gas composition comprising at least CO, CO₂, H₂ and CH₄;
   wherein the gas composition is an off-gas from at least one steam methane reformer and the off-gas from the steam methane reformer is directly brought into contact with the genetically modified homoacetogenic bacterium; and the
   homoacetogenic bacterium is genetically modified to produce acetone from the gas composition. The term ‘gas composition’ herein refers to any mixture of gases. The gas composition is the gas substrate which is the main carbon source for microbial fermentation according to any aspect of the present invention. In particular, the gas composition is a syngas (i.e. comprising CO and H₂). More in particular, the gas composition according to any aspect of the present invention, comprises at least CO, CO₂, H₂ and CH₄. In some examples, N₂, O₂, and H₂S are also present in the gas composition. In particular, N₂, O₂, and H₂S are present in low concentrations compared to the main components of the gas composition, namely CO, CO₂, H₂ and CH₄.

The CO₂ in the gas composition according to any aspect of the present invention may be in the range of 30-80% vol, particularly 35-80, 40-80, 45-80, 50-80, 55-80, 60-80, 65-80, 70-80, 30-75, 35-75, 40-75, 45-75, 50-75, 55-75, 60-75, 65-75, 70-75, 30-70, 35-70, 40-70, 45-70, 50-70, 55-70, 60-70, 65-70, 30-65, 35-65, 40-65, 45-65, 50-65, 55-65, 30-60, 35-60, 40-60, 45-60, 50-60, 30-55, 30-55, 40-55, 45-55, 30-50, 35-50, 40-50, 45-50, 30-45, 35-45, 40-45, 30-40, or 35-40% vol. More in particular, the CO₂ may be about 30, 35, 40, 45, 50, 55, 60, 65, 70, 75 or 80% vol. Even more in particular, the CO₂ may be in the range of 35-65% vol.

The H₂ in the gas composition according to any aspect of the present invention may be in the range of 10-50% vol, particularly, 15-50, 20-50, 25-50, 30-50, 35-50, 40-50, 10-45, 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, or 15-20% vol. More in particular, the H₂ may be about 15, 20, 25, 30, 35, 40, 45 or 50% vol. Even more in particular, the H₂ may be in the range of 20-40% vol.

The CO in the gas composition according to any aspect of the present invention may be in the range of 1-30% vol, particularly, 5-30, 10-30, 15-30, 20-30, 25-30, 1-25, 5-25, 10-25, 15-25, 20-25, 1-20, 5-20, 10-20, 15-20, 1-15, 5-15, 10-15, 1-10, or 5-10% vol. More in particular, the CO may be about 5, 10, 15, 20, 25, 30, % vol. Even more in particular, the H₂ may be in the range of 5-20% vol.

The CH₄ in the gas composition according to any aspect of the present invention is in the range of 0.01-30% vol, particularly, 0.01-25, 0.01-20, 0.01-15, 0.01-10, 0.01-5, 0.01-1, 0.01-0.5, 0.01-0.1, 0.01-0.05, 0.05-30, 0.05-25, 0.05-20, 0.05-15, 0.05-10, 0.05-5, 0.05-1, 0.05-0.5, 0.05-0.1, 0.1-30, 0.1-25, 0.1-20, 0.1-15, 0.1-10, 0.1-5, 0.1-1, 0.1-0.5, 0.5-30, 0.5-25, 0.5-20, 0.5-15, 0.5-10, 0.5-5, 0.5-1, 1-30, 1-25, 1-20, 1-15, 1-10, 1-5, 5-30, 5-25, 5-20, 5-15, 5-10, 10-30, 10-25, 10-20, 10-15, 20-30, or 20-25% vol. More in particular, the CH₄ may be about 0.01, 0.05, 0.1, 0.2, 0.3, 0.4, 0.5, 0.6, 0.7, 0.8, 0.9, 1, 5, 10, 15, 20, 25, 30, % vol. Even more in particular, the CH₄ may be in the range of 0.01-20% vol.

More in particular, the 02 concentration in the gas composition may be may be present at less than 1% by volume of the total amount of gas in the gas composition. In particular, the oxygen may be present at a concentration range of 0.000005 to 2% by volume, at a range of 0.00005 to 2% by volume, 0.0005 to 2% by volume, 0.005 to 2% by volume, 0.05 to 2% by volume, 0.00005 to 1.5% by volume, 0.0005 to 1.5% by volume, 0.005 to 1.5% by volume, 0.05 to 1.5% by volume, 0.5 to 1.5% by volume, 0.00005 to 1% by volume, 0.0005 to 1% by volume, 0.005 to 1% by volume, 0.05 to 1% by volume, 0.5 to 1% by volume, 0.55 to 1% by volume, 0.60 to 1% by volume, particularly at a range of 0.60 to 1.5%, 0.65 to 1%, and 0.70 to 1% by volume. In particular, the acetogenic microorganism is particularly suitable when the proportion of 02 in the gas phase/composition is about 0.00005, 0.0005, 0.005, 0.05, 0.15, 0.5, 0.6, 0.7, 0.8, 0.9, 1, 1.5, 2% by volume in relation to the volume of the gas in the gas composition. A skilled person would be able to use any one of the methods known in the art to measure the volume concentration of oxygen in the gas composition. In particular, the volume of oxygen may be measured using any method known in the art. In one example, a gas phase concentration of oxygen may be measured by a trace oxygen dipping probe from PreSens Precision Sensing GmbH. Oxygen concentration may be measured by fluorescence quenching, where the degree of quenching correlates to the partial pressure of oxygen in the gas phase.

The H₂S concentration in the gas composition according to any aspect of the present invention may be 0.00000001%-0.0001% volume.

The N₂ concentration in the gas composition according to any aspect of the present invention may be 0.1-0.7 vol.-%

The term “about” as used herein refers to a variation within 20 percent. In particular, the term “about” as used herein refers to +/−20%, more in particular, +/−10%, even more in particular, +/−5% of a given measurement or value.

All percentages (%) are, unless otherwise specified, volume percent.

The gas composition according to any aspect of the present invention may be an off- or waste gas from any industrial process. In particular, the off- or waste gas according to any aspect of the present invention may be from at least one steam methane reformer. The off-gas from the steam methane reformer refers to the by-product of the steam methane reforming process, namely the unwanted gas that is produced as a result of the steam methane reforming process. In particular, the off-gas from the steam methane reformer is the source of carbon for the method of acetone production according to any aspect of the present invention.

Steam methane reforming (SMR) is a process in which methane from natural gas is heated, with steam, in the presence of a catalyst, to produce an off- or waste gas comprising mainly of CO, CO₂, H₂ and CH₄ This off- or waste gas may then be used in the method according to any aspect of the present invention to produce acetone. There are at least two reactions that are carried out in the steam methane reformer. These are:

### (1) Steam-Methane Reforming Reaction

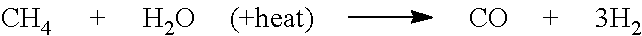

### (2) Water-Gas Shift Reaction

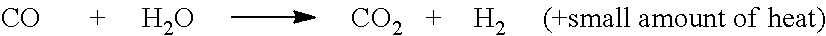

The concentration of the gases (i.e. gas composition) in the off- or waste gas from the steam methane reformer is suitable for direct use in microbial fermentation to produce acetone.

Accordingly, the off- or waste gas from the steam methane reformer may be directly brought into contact with at least one genetically modified homoacetogenic bacteria to produce acetone. In particular, the bacteria is brought directly or immediately in contact with the off- or waste gas from the steam methane reformer without any additional steps, in particular without any purification steps of the off- or waste gas to prepare the gas for use in fermentation. The off- or waste gas from the steam methane reformer is thus suitable for direct use in the fermenter for acetone production. More in particular, the off- or waste gas from the steam methane reformer is brought into direct contact with the bacteria without the necessity of prior pre-treatment or scrubbing.

The term “homoacetogenic bacteria” as used herein is interchangeable with the term ‘acetogenic bacteria’ that refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria used according to any aspect of the present invention is a genetically modified bacteria that is genetically modified to produce acetone from a carbon source, particularly an off- or waste gas from the steam methane reformer. The genetically modified cell is an acetogenic cell that is genetically modified to increase expression of an enzyme that enables the cell to produce acetone from a carbon source, compared to a wild type cell, particularly an off- or waste gas from the steam methane reformer.

The phrase “increased heterologous expression of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures.

Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. In particular, an increase in an activity of an enzyme relative to the wild type cell may be a 10, 15, 20, 25, 30, 35, 40, 45, 50, 55, 60, 65, 70, 75, 80, 85, 90, 95 or 100% more than the wild type cell.

A skilled person would be able to use any method known in the art to genetically modify a cell. Whether or not a nucleic acid molecule, polypeptide, more specifically an enzyme used according to any aspect of the present invention, is recombinant or not has not necessarily implications for the level of its expression. However, in one example one or more recombinant nucleic acid molecules, polypeptides or enzymes used according to any aspect of the present invention may be overexpressed. The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art.

DE-A-100 31 999 gives a general survey of the possibilities for increasing the enzyme activity in cells as exemplified by pyruvate carboxylase, which is inserted hereby as a reference and whose disclosure content with respect to the possibilities for increasing the enzyme activity in cells forms a part of the disclosure of the present invention.

The expression of the above and all subsequently mentioned enzymes or genes is detectable with the aid of 1- and 2-dimensional protein gel separation and subsequent optical identification of the protein concentration in the gel using appropriate analytical software. If the increase in an enzyme activity is based exclusively on an increase in the expression of the corresponding gene, the quantification of the increase in the enzyme activity can be determined in a simple manner by a comparison of the 1- or 2-dimensional protein separations between wild-type and genetically modified cell. A customary method for the preparation of the protein gels in the case of coryneforme bacteria and for the identification of the proteins is the procedure described by Hermann et al. (Electrophoresis, 22: 1712.23 (2001)). The protein concentration can likewise be analyzed by Western Blot hybridization using an antibody specific for the protein to be detected (Sambrook et al., Molecular Cloning: a laboratory manual, 2nd Ed. Cold Spring Harbor Laboratory Press, Cold Spring Harbor, N.Y. USA, 1989) and subsequent optical analysis using appropriate software for the concentration determination (Lohaus and Meyer (1989) Biospektrum, 5: 32-39; Lottspeich (1999) Angewandte Chemie 111: 2630-2647). The activity of DNA-binding proteins can be measured by means of DNA band shift assays (also called gel retardation) (Wilson et al. (2001) Journal of Bacteriology, 183: 2151-2155). The action of DNA-binding proteins on the expression of other genes can be detected by various well-described methods of the reporter gene assay (Sambrook et al., Molecular Cloning: a laboratory manual, 2nd Ed. Cold Spring Harbor Laboratory Press, Cold Spring Harbor, N.Y. USA, 1989). The intracellular enzymatic activities can be determined according to various described methods (Donahue et al. (2000) Journal of Bacteriology 182 (19): 5624-5627; Ray et al. (2000) Journal of Bacteriology 182 (8): 2277-2284; Freedberg et al. (1973) Journal of Bacteriology 115 (3): 816-823). If in the following embodiments no practical methods are indicated for the determination of the activity of a certain enzyme, the determination of the increase in the enzyme activity and also the determination of the decrease of an enzyme activity preferably take place by means of the methods described in Hermann et al., Electophoresis, 22: 1712-23 (2001), Lohaus et al., Biospektrum 5 32-39 (1998), Lottspeich, Angewandte Chemie 111: 2630-2647 (1999) and Wilson et al., Journal of Bacteriology 183: 2151-2155 (2001).

If the increase in the enzyme activity is accomplished by mutation of the endogenous gene, such mutations can be randomly produced either by conventional methods, such as, for example, by UV irradiation or by mutagenic chemicals, or selectively by means of genetic engineering methods such as deletion(s), insertion(s) and/or nucleotide exchange(s). Modified cells are obtained by these mutations. Particularly preferred mutants of enzymes are in particular also those enzymes that are no longer feedback-, product- or substrate inhibitable or are so to a reduced degree at least in comparison to the wild-type enzyme.

If the increase in the enzyme activity is accomplished by increase in the synthesis of an enzyme, the copy number of the corresponding genes is increased or the promoter and regulation region or the ribosome binding site, which is situated upstream of the structural gene, is mutated. Expression cassettes, which are incorporated upstream of the structural gene, act in the same manner. It is additionally possible, by means of inducible promoters, to increase the expression at any desired point in time. In addition, however, also “enhancers” can be assigned to the enzyme gene as regulatory sequences, which likewise bring about increased gene expression by means of an improved interaction between RNA polymerase and DNA. As a result of measures for the prolongation of the lifetime of the mRNA, the expression is likewise improved. Furthermore, by prevention of the degradation of the enzyme protein the enzyme activity is likewise increased. The genes or gene constructs are present here either in plasmids having a different copy number or are integrated and amplified in the chromosome. Alternatively, an overexpression of the genes concerned can furthermore be achieved by modification of the media composition and culture management. The person skilled in the art finds directions for this, inter alia, in Martin et al. (Bio/Technology 5, 137-146 (1987)), in Guerrero et al. (Genes 138, 35-41 (1994)), Tsuchiya and Morinaga (Bio/Technology 6, 428-430 (1988)), in Eikmanns et al. (Genes 102, 93-98 (1991)), in EP-A-0 472 869, in U.S. Pat. No. 4,601,893, in Schwarzer and Puhler (Bio/Technology 9, 84-87 (1991)), in Reinscheid et al. (Applied and Environmental Microbiology 60, 126-132 (1994)), in LaBarre et al. (Journal of Bacteriology 175, 1001-1007 (1993)), in WO-A96/15246, in Malumbres et al. (Genes 134, 15-24 (1993)), in JP-A-10-229891, in Jensen and Hammer (Biotechnology and Bioengineering 58, 191-195 (1998)) and in known textbooks of genetics and molecular biology. The measures described above likewise lead, like the mutations, to genetically modified cells Episomal plasmids, for example, are employed for increasing the expression of the respective genes.

Suitable plasmids or vectors are in principle all embodiments available for this purpose to the person skilled in the art. Such plasmids and vectors can be taken, for example, from the brochures of the companies Novagen, Promega, New England Biolabs, Clontech or Gibco BRL. Further preferred plasmids and vectors can be found in: Glover, D. M. (1985) DNA cloning: a practical approach, Vol. I-III, IRL Press Ltd., Oxford; Rodriguez, R. L. and Denhardt, D. T (eds) (1988) Vectors: a survey of molecular cloning vectors and their uses, 179-204, Butterworth, Stoneham; Goeddel, D. V. (1990) Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York. The plasmid vector, which contains the gene to be amplified, is then converted to the desired strain by conjugation or transformation. The method of conjugation is described, for example, in Schafer et al., Applied and Environmental Microbiology 60: 756-759 (1994). Methods for transformation are described, for example, in Thierbach et al., Applied Microbiology and Biotechnology 29: 356-362 (1988), Dunican and Shivnan, Bio/Technology 7: 1067-1070 (1989) and Tauch et al., FEMS Microbiology Let-ters 123: 343-347 (1994). After homologous recombination by means of a “cross-over” event, the resulting strain contains at least two copies of the gene concerned. In particular, to increase the activity of at least one enzyme E₁, E₂ and/or E₃:
a) at least one promoter which is operably linked to a gene encoding any one of the enzymes E₁, E₂ and/or E₃ in the suitable chromosome of the cell, or
b) at least one expression vector to increase the copy number of gene(s) encoding any one of the enzymes E₁, E₂ and/or E₃in the cell, or
c) combination of (a) and (b).

According to any aspect of the present invention, the cell may be genetically modified so that in a defined time interval, within 2 hours, in particular within 8 hours or 24 hours, it forms at least twice, especially at least 10 times, at least 100 times, at least 1000 times or at least 10000 times more ketones than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (lipid with general formula II or I) in the nutrient medium.

In the same context, the phrase “decreased activity and/or expression of an enzyme Ex” used with reference to any aspect of the present invention may be understood as meaning an activity decreased by a factor of at least 0.5, particularly of at least 0.1, more particularly of at least 0.01, even more particularly of at least 0.001 and most particularly of at least 0.0001. The phrase “decreased activity” also comprises no detectable activity (“activity of zero”). The decrease in the activity of a certain enzyme can be effected, for example, by selective mutation or by other measures known to the person skilled in the art for decreasing the activity of a certain enzyme. In particular, the person skilled in the art finds instructions for the modification and decrease of protein expression and concomitant lowering of enzyme activity by means of interrupting specific genes, for example at least in Dubeau et al. 2009. Singh & Röhm. 2008., Lee et al., 2009 and the like. The decrease in the enzymatic activity in a cell according to any aspect of the present invention may be achieved by modification of a gene comprising one of the nucleic acid sequences, wherein the modification is selected from the group comprising, consisting of, insertion of foreign DNA in the gene, deletion of at least parts of the gene, point mutations in the gene sequence, RNA interference (siRNA), antisense RNA or modification (insertion, deletion or point mutations) of regulatory sequences, such as, for example, promoters and terminators or of ribosome binding sites, which flank the gene. In particular, to decrease the activity of an enzyme in a cell, the cell may comprise
a) a foreign DNA in the gene encoding the enzyme;
b) a deletion of at least one part of the gene encoding the enzyme,
c) at least one point mutation, RNA interference (siRNA), antisense RNA in the gene and/or regulatory sequences of the gene encoding the enzyme; or
d) combinations of (d), (e) and (f).

The expression of the enzymes and genes mentioned above, and all mentioned below is determinable by means of 1- and 2-dimensional protein gel separation followed by optical identification of the protein concentration in the gel with appropriate evaluation software.

The homoacetogenic bacterium according to any aspect of the present invention may be genetically modified to:
increase expression relative to its wild-type of at least one of the following enzymes
thiolase (ThIA, E₁) (E.C.2.3.1.9),
CoA transferase (CtfAB, E₂) (EC 2.8.3.8) and/or
acetoacetate decarboxylase (Adc, E₃) (EC 4.1.1.4); and/or
   decrease expression relative to its wild-type of at least:
secondary alcohol dehydrogenase (sAdh, E₄) (EC 1.1.1.1).

In one example, the cell may be genetically modified to increase expression relative to its wild-type cell of enzymes E₁, E₂, and E₃ and genetically modified to decrease expression relative to its wild-type cell of enzyme E₄.

In particular, E₁ may be capable of catalyzing the conversion of acetyl-CoA to acetoacetyl-CoA. E₁ may be an acetoacetyl-CoA thiolase also known as an acetyl-Coenzyme A acetyltransferase. Acetoacetyl-CoA thiolase enzymes include the gene products of atoB from E. coli (Martin et al., 2003) with accession number NP_416728, thiolase derived from C. acetobutylicum. More in particular, E₁ may comprise an amino acid sequence that has 50, 55, 60, 65, 70, 75, 80, 85, 90, 95 or 100% sequence identity to SEQ ID NO: 2. Even more in particular, the cell according to any aspect of the present invention may be genetically modified to comprise the sequence of SEQ ID NO:1.

A skilled person may be capable of identifying other thiolases that may play the role of E₁. In particular, the a skilled person may be capable of assessing whether a functionally equivalent variant has substantially the same function as the nucleic acid or polypeptide of which it is a variant using any number of known methods. In one example, the methods outlined in Wiesenborn et al 1988, Wiesenborn et al, 1989, Peterson and Bennet, 1990, Ismail et al., 1993, de la Plaza et al, 2004 or may be used to assess the enzyme activity of E₁.

E₂ may be an acetoacetate CoA transferase (EC 2.8.3.9). The acetoacetate CoA transferase conserves the energy stored in the CoA-ester bond. These enzymes either naturally exhibit the desired acetoacetyl-CoA transferase activity or they can be engineered via directed evolution to accept acetetoacetyl-CoA as a substrate with increased efficiency. In particular, such enzymes, may also be capable of catalyzing the conversion of 3-hydroxybutyryl-CoA to 3-hydroxybutyrate via a transferase mechanism. Examples of E₂ may include the CoA transferase from E. coli with accession number P76459.1 or P76458.1 (Hanai et al., 2007), ctfAB from C. acetobutylicum with accession number NP_149326.1 or NP_149327.1 (Jojima et al., 2008), ctfAB from Clostridium saccharoperbutylacetonicum with accession number AAP42564.1 or AAP42565.1 (Kosaka et al., 2007) and the like. In particular, E₂ may also be selected from the group consisting of the gene products of cat1, cat2, and cat3 of Clostridium kluyveri with accession number P38946.1, P38942.2, and EDK35586.1 respectively (Seedorf et al., 2008; Sohling and Gottschalk, 1996), transferase products of Trichomonas vaginalis with accession number XP_001330176 (van Grinsven et al., 2008), Trypanosoma brucei with accession number XP_828352 (Riviere et al, 2004), Fusobacterium nucleatum (Barker et al., 1982), Clostridium SB4 (Barker et al., 1978), Clostridium acetobutylicum (Wiesenborn et al., 1989), FN0272 and FN0273 with accession numbers NP_603179.1 and NP_603180.1 respectively (Kapatral et al., 2002), homologs in Fusobacterium nucleatum such as FN1857 and FN1856 with accession numbers NP_602657.1 and NP_602656.1 (Kreimeyer, et al., 2007), transferase products of Porphyrmonas gingivalis with accession number NP_905281.1 or NP_905290.1 and Thermoanaerobacter tengcongensis with accession number NP_622378.1 or NP_622379.1 (Kreimeyer, et al., 2007). More in particular, E₂ may comprise an amino acid sequence that has 50, 55, 60, 65, 70, 75, 80, 85, 90, 95 or 100% sequence identity to SEQ ID NO: 4 or 6. In particular, E₂ may comprise an amino acid sequence SEQ ID NO: 4 and 6. E₂ may comprise a nucleotide sequence that has 50, 55, 60, 65, 70, 75, 80, 85, 90, 95 or 100% sequence identity to SEQ ID NO: 3 or 5. More in particular, E₂ may comprise a nucleotide sequence that has 50, 55, 60, 65, 70, 75, 80, 85, 90, 95 or 100% sequence identity to SEQ ID NO: 3 and 5.

The expression of E₂ may be measured using any method known in the art. In particular, the increase in expression of E₂ may be measured by determining the amount of final product obtained in the presence of the enzyme and comparing the result to the amount of final product obtained in the absence of the enzyme E₂. In another example, the expression of E₂ may be determined by determining the amount of E₂ protein expressed in the resulting medium. In one example the expression of E₂ may be measured using the method disclosed in Charrier C., 2006.

E₃ may be an acetoacetate decarboxylase (Adc; EC 4.1.1.4). Acetoacetate decarboxylases used according to any aspect of the present invention are selected from the list NP_149328.1, YP_001310906.1 and CAQ57986.1 and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₃. More in particular, E₃ may comprise an amino acid sequence that has 50, 55, 60, 65, 70, 75, 80, 85, 90, 95 or 100% sequence identity to SEQ ID NO: 7. In particular, E₂ may comprise an amino acid sequence SEQ ID NO: 7.

A method for determining the activity is described in Daniel et al. Appl. Environ. Microbiol. 1990, pp. 3491-3498 Vol. 56, No. 11.

E₄ may be an alcohol dehydrogenase. “Alcohol dehydrogenases” may include alcohol dehydrogenases which are capable of catalysing the conversion of ketones (such as acetone) to secondary alcohols (such as isopropanol), or vice versa. Such alcohol dehydrogenases include secondary alcohol dehydrogenases and primary alcohol dehydrogenases. A “secondary alcohol dehydrogenase” is one which can convert ketones (such as acetone) to secondary alcohols (such as isopropanol), or vice versa. A “primary alcohol dehydrogenase” is one which can convert aldehydes to primary alcohols, or vice versa; however, a number of primary alcohol dehydrogenases are also capable of catalysing the conversion of ketones to secondary alcohols, or vice versa. These alcohol dehydrogenases may also be referred to as “primary-secondary alcohol dehydrogenases”. Membrane-bound, flavin-dependent alcohol dehydrogenases of the Pseudomonas putida GPO1 AIkJ type exist which use flavor cofactors instead of NAD+. A further group comprises iron-containing, oxygen-sensitive alcohol dehydrogenases which are found in bacteria and in inactive form in yeast. Another group comprises NAD+-dependent alcohol dehydrogenases, including zinc-containing alcohol dehydrogenases, in which the active center has a cysteine-coordinated zinc atom, which fixes the alcohol substrate. In one example, under the expression “alcohol dehydrogenase”, as used herein, it is understood to mean an enzyme which oxidizes an aldehyde or ketone to the corresponding primary or secondary alcohol. In particular, the alcohol dehydrogenase according to any aspect of the present invention may be an NAD+-dependent alcohol dehydrogenase, i.e. an alcohol dehydrogenase which uses NAD+ as a cofactor for oxidation of the alcohol or NADH for reduction of the corresponding aldehyde or ketone. In the most preferred embodiment, the alcohol dehydrogenase is an NAD+-dependent, zinc-containing alcohol dehydrogenase. Examples of suitable NAD+-dependent alcohol dehydrogenases may include the alcohol dehydrogenase A from Rhodococcus ruber (database code AJ491307.1) or a variant thereof. Further examples comprising the alcohol dehydrogenases of Ralstonia eutropha (ACB78191.1), Lactobacillus brevis (YP_795183.1), Lactobacillus kefiri (ACF95832.1), from horse liver, of Paracoccus pantotrophus (ACB78182.1) and Sphingobium yanoikuyae (EU427523.1) and also the respective variants thereof. In one example, the expression “NAD(P)+-dependent alcohol dehydrogenase”, as used herein, designates an alcohol dehydrogenase which is NAD+- and/or NADP+-dependent.

In one example, E₄ may be a secondary alcohol dehydrogenase or selected from other alcohol dehydrogenases or equivalently aldehyde reductases and can also serve as candidates for 3-hydroxybutyraldehyde reductase. T. E₄ may be the product of the gene selected from the group consisting of adhl from Geobacillus thermoglucosidasius with accession number AAR91477.1 (Jeon et al., 2008), SADH from C. beijerinckii the alcohol dehydrogenases disclosed in Tani et al., 2000 with accession number BAB122273.1 may be used as E₄. E₄ may also be selected from the group consisting of ADH2 from Saccharomyces cerevisiae with accession number NP_014032.1 (Atsumi et al., 2008), yqhD from E. coli with accession number NP_417484.1 (Sulzenbacher et al., 2004 and Perez et al., 2008), bdh I and bdh II from C. acetobutylicum with accession number NP_349892.1 and NP_349891.1 respectively (Walter et al., 1992), and ADH1 from Zymomonas mobilis with accession number YP_162971.1 (Kinoshita et al., 1985).

More in particular, E₄ may be a secondary alcohol dehydrogenase. Even more in particular, E₄ may be a secondary alcohol dehydrogenase from C. beijerinckii. E₄ may comprise an amino acid sequence that has 50, 55, 60, 65, 70, 75, 80, 85, 90, 95 or 100% sequence identity to SEQ ID NO: 8. E₄ may comprise a nucleotide sequence that has 50, 55, 60, 65, 70, 75, 80, 85, 90, 95 or 100% sequence identity to SEQ ID NO: 7.

The cell according to any aspect of the invention, in order to express significantly reduced levels of adc, may be genetically modified to remove expression of adc which may be achieved by using standard recombinant DNA technology known to the person skilled in the art. The gene sequences respectively responsible for production of adc may be inactivated or partially or entirely eliminated. Thus, the cell according to any aspect of the present invention expresses reduced or undetectable levels of adc or expresses functionally inactive adc.

In one example, the cell according to any aspect of the present invention may express E₄ naturally and may be genetically modified to reduce the expression of E₄ in the cell to about 0% or undetectable levels relative to the wild type cell. In another example, the cell according to any aspect of the present invention has about 0% or undetectable levels of expression of E₄ in its' wild type form. In particular, when the cell according to any aspect of the present invention has undetectable expression of the enzyme E₄.

Any accession number used in the application refers to the respective sequence from the Genbank database run by the NCBI, wherein the release referred to is the one available online on the 30^{th} March 2015.

The expression of E₄ may be measured using any method known in the art. In particular, the increase in expression of E₄ may be measured by determining the amount of final product obtained in the presence of the enzyme and comparing the result to the amount of final product obtained in the absence of the enzyme E₄. In another example, the expression of E₄ may be determined by determining the amount of E₄ protein expressed in the resulting medium. In one example the expression of E₄ may be measured using the method disclosed in Ismaiel, A.A. (1993).

In particular, the acetogenic bacteria that is genetically modified according to any aspect of the present invention is selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061), Clostridium carboxidivorans (DSM 15243), Clostridium drakei (ATCC BAA-623), Clostridium formicoaceticum (DSM 92), Clostridium glycolicum (DSM 1288), Clostridium ljungdahlii (DSM 13528), Clostridium mayombei (DSM 6539), Clostridium methoxybenzovorans (DSM 12182), Clostridium ragsdalei (DSM 15248), Clostridium scatologenes (DSM 757), Clostridium species A TCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 3222), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ERI2, Clostridium ljungdahlii COL and Clostridium ljungdahlii 0-52 may be used in the conversion of synthesis gas to hexanoic acid. These strains for example are described in WO 98/00558, WO 00/68407, ATCC 49587, ATCC 55988 and ATCC 55989. Even more in particular, the homoacetogenic bacteria may be selected from Clostridium family.

The homoacetogenic bacterium is selected from the group consisting of Clostridium autoethanogenum (DSM 10061, DSM 19630 and DSM 23693), Clostridium ljungdahlii (DSM 13528), Clostridium carboxidivorans (DSM 15243), Acetobacterium woodii (DSM 1030), Clostridium ragsdalei (DSM 15248), Clostridium drakei (ATCC BAA-623), Moorella thermoacetica (DSM 521), Moorella thermoautotrophica (DSM 1974), Sporomusa silvacetica (DSM 10669), and Alkalibaculum bacchi (DSM 22112). In particular, the homoacetogenic bacteria may be selected from the group consisting of Clostridium autoethanogenum, Clostridium ljungdahlii and Clostridium carboxidivorans. In one example, for acetone production a genetically modified C. autoethanogenum may be used. In another example, for acetone production a genetically modified Clostridium ljungdahlii may be used.

The method according to any aspect of the present invention, comprising a step of
(a) receiving a stream of the off or waste gas from the steam methane reformer.

The term “stream” as used herein refers to a flow of material into, through and away from one or more stages of a process, for example, the material that is fed to a bioreactor where fermentation is to be carried out. The composition of the stream may vary as it passes through particular stages.

For example, as a stream enters the bioreactor, it may comprise a significant amount of CO, CO₂, H₂ and CH₄ and low concentrations of N₂, O₂, and H₂S. When the stream passes through the bioreactor, the CO, CO₂ and H₂ content of the stream may decrease.

The terms “fermentation process” or “fermentation reaction” or “microbial fermentation” and the like, as used herein, are intended to encompass both the growth phase and product biosynthesis, in particular acetone, phase of the method according to any aspect of the present invention.

There may be a further step of purification of the acetone. In particular, acetone may be directly purified from the crude product stream by distillation following the fermentation process.

According to another aspect of the present invention, there is provided an apparatus for producing acetone from a gaseous composition by microbial fermentation, the apparatus comprises:
(i) a source for the gaseous composition for continuously providing an off or waste gas stream from a steam methane reformer comprising at least CO, CO₂, H₂ and CH₄,
(ii) an inlet on a bioreactor for receiving the off or waste gas from the steam methane reformer,
(iii) a bioreactor comprising a culture of a homoacetogenic bacteria,
(iv) an outlet on the bioreactor for exiting of the off or waste gas after contact with the homoacetogenic bacteria wherein the source provides the gaseous composition coming directly from the steam methane reformer.

According to a further aspect of the present invention, there is provided a use of an off or waste gas from a steam methane reformer for production of acetone, wherein the off or waste gas comprises at least CO, CO₂, H₂ and CH₄ and the off or waste gas is brought into contact with at least one homoacetogenic bacteria.

According to yet a further aspect of the present invention, there is provided a steam methane reformer adapted to produce acetone by microbial fermentation of off or waste gas(es) from the steam methane reformer.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

High Production of Acetone by Clostridium ljungdahlii with Off-Gas from a SMR

For the biotransformation of hydrogen, carbon monoxide and carbon dioxide to acetone a genetically modified homoacetogenic bacterium Clostridium ljungdahlii (GMO from EP2421960B1) is cultivated on off gas from a steam methane reformer. All cultivation steps are carried out under anaerobic conditions in pressure-resistant glass bottles that can be closed airtight with a butyl rubber stopper or in stirred tank stainless steel bench top bioreactors.

For the preculture, 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L COCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9 H₂O are inoculated with 2,5 μL of a frozen cryo stock of C. ljungdahlii GMO. The chemolithoautotrophic cultivation is carried out in a 1 L pressure-resistant glass bottle at 37° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 66 h. The gas is discharged into the medium through a sparger with a pore size of 10 μm, which is mounted in the center of the reactors. Culturing is carried out with no pH control.

For the main culture, as many cells from the preculture as necessary for an OD_{600 nm} of 0.1 are transferred from the preculture in fresh 500 mL medium. For the main culture LM33 mineral medium (pH=5.8, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO₄×7 H₂O, 5 mg/L MnSO₄×H₂O, 1 mg/L FeSO₄×7 H₂O, 8 mg/L Fe(SO₄)₂(NH₄)₂×6 H₂O, 2 mg/L COCl₂×6 H₂O, 2 mg/L ZnSO₄×7 H₂O, 200 μg/L CuCl₂×2 H₂O, 200 μg/L KAI(SO₄)₂×12 H₂O, 3 mg/L H₃BO₃, 300 μg/L Na₂MoO₄×2 H₂O, 200 μg/L Na₂SeO₃, 200 μg/L NiCl₂×6 H₂O, 200 μg/L Na₂WO₄×6 H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B12; 500 μg/L p-aminobenzoate; 500 μg/L lipoic acid, 10 mg/L FeCl₃, aerated for 30 min with the steam methane reformer off gas mixture), with additional 500 mg/L L-cysteine-hydrochloride is used.

The chemolithoautotrophic cultivation is carried out in a 1 L pressure-resistant glass bottle at 37° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 164 h. The gas is discharged into the medium through a sparger with a pore size of 10 μm, which is mounted in the center of the reactors. The pH is hold at 5.0 by automatic addition of 100 g/L NaOH solution by a Titrino pH control system (Methrom, Switzerland). During cultivation several 5 mL samples are taken to determinate OD_{600 nm}, pH und product formation. The determination of the product concentrations is performed by semiquantitative 1H-NMR spectroscopy. As an internal quantification standard sodium trimethylsilylpropionate (T(M)SP) is used.

During the main cultivation in LM33 medium the concentration of ethanol increases from 0 g/L to >2 g/L, the concentration of acetate increases from 0 g/L to >0.5 g/L and the concentration of acetone increases from 0 g/L to >2 g/L. The OD_{600 nm} reaches a maximum of >2 after 100 h of cultivation.

### Example 2

Production of acetone by Acetobacterium woodii with off-gas from a SMR For the biotransformation of hydrogen, carbon monoxide and carbon dioxide to acetone a genetically modified homoacetogenic bacterium Acetobacterium woodii (GMO from EP2421960B1) is cultivated on off gas from a steam methane reformer. All cultivation steps are carried out under anaerobic conditions in pressure-resistant glass bottles that can be closed airtight with a butyl rubber stopper or in stirred tank stainless steel bench top bioreactors.

For the preculture 500 ml medium (DSMZ135-medium: pH=8.2; 1.0 g/L NH₄Cl; 0.33 g/L KH₂PO₄; 0.45 g/L K₂HPO₄; 0.1 g/L MgSO₄×7 H₂O; 2.0 g/L yeast extract; 500 mg/L L-cysteine-HCL×H₂O; 500 mg/L Na₂S×9 H₂O; 10 g/L NaHCO₃; 30 mg/L nitrilotriacetic acid; 60 mg/L MgSO₄×7 H₂O; 10 mg/L MnSO₄×H₂O; 20 mg/L NaCl; 2 mg/L FeSO₄×7 H₂O; 3.6 mg/L CoSO₄×7 H₂O; 2 g/L CaCl₂×2 H₂O; 3.6 mg/L ZnSO₄×7 H₂O; 0.2 mg/L CuSO₄×7 H₂O; 0.4 mg/L KAI(SO₄)₂×12 H₂O; 0.2 mg/L H₃BO₃; 0.2 mg/L Na₂MoO₄×2 H₂O; 0.6 mg/L NiCl₂×6 H₂O; 6 μg/L Na₂SeO₃×5 H₂O; 40 μg/L d-biotin; 40 μg/L folic acid; 200 μg/L pyridoxine-HCl; 100 μg/L thiamine-HCl×H₂O; 100 μg/L riboflavin; 100 μg/L nicotinic acid; 100 μg/L Ca-pantothenate; 2 μg/L vitamin B12; 100 μg/L p-aminobenzoate; 100 μg/L lipoic acid) are inoculated with 2,5 μL of a frozen cryo stock of A. woodii GMO. The chemolithoautotrophic cultivation is carried out in a 1 L pressure-resistant glass bottle at 30° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 66 h. The gas is discharged into the medium through a sparger with a pore size of 10 μm, which is mounted in the center of the reactors. Culturing is carried out with no pH control.

For the main culture, as many cells from the preculture as necessary for an OD_{600 nm} of 0.1 are transferred from the preculture in fresh 500 mL medium. For the main culture also DSMZ135 medium (aerated for 30 min with the steam methane reformer off gas mixture) is used. The chemolithoautotrophic cultivation is carried out in a 1 L pressure-resistant glass bottle at 30° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 164 h. The gas is discharged into the medium through a sparger with a pore size of 10 μm, which is mounted in the center of the reactors. The pH is hold at 7.5 by automatic addition of 100 g/L NaOH solution by a Titrino pH control system (Methrom, Switzerland). During cultivation several 5 mL samples are taken to determinate OD_{600 nm}, pH und product formation. The determination of the product concentrations is performed by semiquantitative 1H-NMR spectroscopy. As an internal quantification standard sodium trimethylsilylpropionate (T(M)SP) is used.

During the main cultivation in DSMZ135 medium the concentration of acetate increases from 0 g/L to >4 g/L, the concentration of acetone increases from 0 g/L to >0.2 g/L and the concentration of isopropanol increases from 0 g/L to >0.4 g/L. The OD_{600 nm} reaches a maximum of >2 after 100 h of cultivation.

As can be seen here, compared to Example 1, A. woodii can produce acetone from SMR off-gases. However, less acetone is produced compared to Example 1.

### Example 3

Low Production of Acetone by Clostridium ljungdahlii on Synthesis Gas

For the biotransformation of hydrogen and carbon dioxide to acetone a genetically modified homoacetogenic bacterium Clostridium ljungdahlii (GMO from EP2421960B1) is cultivated on a synthesis gas mixture with hydrogen, carbon dioxide and methane. All cultivation steps are carried out under anaerobic conditions in pressure-resistant glass bottles that can be closed airtight with a butyl rubber stopper or in stirred tank stainless steel bench top bioreactors.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L COCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9 H₂O are inoculated with 2,5 μL of a frozen cryo stock of C. ljungdahlii GMO. The chemolithoautotrophic cultivation is carried out in a 1 L pressure-resistant glass bottle at 37° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 66 h. The gas is discharged into the medium through a sparger with a pore size of 10 μm, which is mounted in the center of the reactors. Culturing is carried out with no pH control.

For the main culture, as many cells from the preculture as necessary for an OD_{600 nm} of 0.1 are transferred from the preculture in fresh 500 mL medium. For the main culture LM33 mineral medium (pH=5.8, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO₄×7 H₂O, 5 mg/L MnSO₄×H₂O, 1 mg/L FeSO₄×7 H₂O, 8 mg/L Fe(SO₄)₂(NH₄)₂×6 H₂O, 2 mg/L COCl₂×6 H₂O, 2 mg/L ZnSO₄×7 H₂O, 200 μg/L CuCl₂×2 H₂O, 200 μg/L KAI(SO₄)₂×12 H₂O, 3 mg/L H₃BO₃, 300 μg/L Na₂MoO₄×2 H₂O, 200 μg/L Na₂SeO₃, 200 μg/L NiCl₂×6 H₂O, 200 μg/L Na₂WO₄×6 H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B12; 500 μg/L p-aminobenzoate; 500 μg/L lipoic acid, 10 mg/L FeCl₃, aerated for 30 min with the H₂/CO₂/CH₄ mixture for the main culture), with additional 500 mg/L L-cysteine-hydrochloride is used.

The chemolithoautotrophic cultivation is carried out in a 1 L pressure-resistant glass bottle at 37° C., 150 rpm and a ventilation rate of 1 L/h with a gas mixture with hydrogen, carbon dioxide and methane (30% CO₂, 60% H₂, 10% CH₄) in an open water bath shaker for 164 h. The gas is discharged into the medium through a sparger with a pore size of 10 μm, which is mounted in the center of the reactors. The pH is hold at 5.0 by automatic addition of 100 g/L NaOH solution by a Titrino pH control system (Methrom, Switzerland). During cultivation several 5 mL samples are taken to determinate OD_{600 nm}, pH und product formation. The determination of the product concentrations is performed by semiquantitative 1H-NMR spectroscopy. As an internal quantification standard sodium trimethylsilylpropionate (T(M)SP) is used.

During the main cultivation in LM33 medium the concentration of ethanol increases from 0 g/L to ~1 g/L, the concentration of acetate increases from 0 g/L to ~0.5 g/L and the concentration of acetone increases from 0 g/L to~1 g/L. The OD_{600 nm} reaches a maximum of ~1 after 100 h of cultivation.

In this example, C. ljungdahlii has not only lower growth but also produces less acetone with another carbon source-synthesis gas mixture without CO compared to example 1.

### Example 4

### Low Production of Acetone by Acetobacterium Woodii on Synthesis Gas

For the biotransformation of hydrogen, carbon monoxide and carbon dioxide to acetone a genetically modified homoacetogenic bacterium Acetobacterium woodii (GMO from EP2421960B1) is cultivated on a synthesis gas mixture with hydrogen, carbon dioxide, carbon monoxide and methane. All cultivation steps are carried out under anaerobic conditions in pressure-resistant glass bottles that can be closed airtight with a butyl rubber stopper or in stirred tank stainless steel bench top bioreactors.

For the preculture 500 ml medium (DSMZ135-medium: pH=8.2; 1.0 g/L NH₄Cl; 0.33 g/L KH₂PO₄; 0.45 g/L K₂HPO₄; 0.1 g/L MgSO₄×7 H₂O; 2.0 g/L yeast extract; 500 mg/L L-cysteine-HCL×H₂O; 500 mg/L Na₂S×9 H₂O; 10 g/L NaHCO₃; 30 mg/L nitrilotriacetic acid; 60 mg/L MgSO₄×7 H₂O; 10 mg/L MnSO₄×H₂O; 20 mg/L NaCl; 2 mg/L FeSO₄×7 H₂O; 3.6 mg/L CoSO₄×7 H₂O; 2 g/L CaCl₂×2 H₂O; 3.6 mg/L ZnSO₄×7 H₂O; 0.2 mg/L CuSO₄×7 H₂O; 0.4 mg/L KAI(SO₄)₂×12 H₂O; 0.2 mg/L H₃BO₃; 0.2 mg/L Na₂MoO₄×2 H₂O; 0.6 mg/L NiCl₂×6 H₂O; 6 μg/L Na₂SeO₃×5 H₂O; 40 μg/L d-biotin; 40 μg/L folic acid; 200 μg/L pyridoxine-HCl; 100 μg/L thiamine-HCl×H₂O; 100 μg/L riboflavin; 100 μg/L nicotinic acid; 100 μg/L Ca-pantothenate; 2 μg/L vitamin B12; 100 μg/L p-aminobenzoate; 100 μg/L lipoic acid) are inoculated with 2,5 μL of a frozen cryo stock of A. woodii GMO. The chemolithoautotrophic cultivation is carried out in a 1 L pressure-resistant glass bottle at 30° C., 150 rpm and a ventilation rate of 1 L/h with a steam methane reformer off gas mixture (49% CO₂, 30% H₂, 10,2% CO, 10,2% CH₄, 0.7% N₂, 100 ppb H₂S, 10 ppm O₂) in an open water bath shaker for 66 h. The gas is discharged into the medium through a sparger with a pore size of 10 μm, which is mounted in the center of the reactors. Culturing is carried out with no pH control.

For the main culture, as many cells from the preculture as necessary for an OD_{600 nm} of 0.1 are transferred from the preculture in fresh 500 mL medium. For the main culture also DSMZ135 medium (aerated for 30 min with the H₂/CO₂/CO/CH₄ mixture for the main culture) is used. The chemolithoautotrophic cultivation is carried out in a 1 L pressure-resistant glass bottle at 30° C., 150 rpm and a ventilation rate of 1 L/h with a gas mixture with hydrogen, carbon dioxide, carbon monoxide and methane (22% CO₂, 25% CO, 43% H₂, 10% CH₄) in an open water bath shaker for 164 h. The gas is discharged into the medium through a sparger with a pore size of 10 μm, which is mounted in the center of the reactors. The pH is hold at 7.5 by automatic addition of 100 g/L NaOH solution by a Titrino pH control system (Methrom, Switzerland). During cultivation several 5 mL samples are taken to determinate OD_{600 nm}, pH und product formation. The determination of the product concentrations is performed by semiquantitative 1H-NMR spectroscopy. As an internal quantification standard sodium trimethylsilylpropionate (T(M)SP) is used.

During the main cultivation in DSMZ135 medium the concentration of acetate increases from 0 g/L to ~2 g/L, the concentration of acetone increases from 0 g/L to ~0.1 g/L and the concentration of isopropanol increases from 0 g/L to~0.2 g/L. The OD_{600 nm} reaches a maximum of ~1 after 100 h of cultivation.

In this example, A. woodii using synthesis gas mixture with high CO content results in lower cell growth and lower acetone production compared to example 2.

1. A method of producing acetone from a gas composition by microbial fermentation, the method comprising:
contacting directly at least one genetically modified homoacetogenic bacterium with the gas composition, the gas composition comprising at least CO, CO₂, H₂ and CH₄;
wherein the gas composition is an off-gas from at least one steam methane reformer and the off gas from the steam methane reformer is directly brought into contact with the genetically modified homoacetogenic bacterium; and the
homoacetogenic bacterium is genetically modified to produce acetone from the gas composition.

2. The method according to claim 1, wherein the homoacetogenic bacterium is genetically modified to:
increase expression relative to its wild-type of at least one of the following enzymes:
thiolase (ThlA) (E.C.2.3.1.9),
CoA transferase (CtfAB) (EC 2.8.3.8) and/or
acetoacetate decarboxylase (Adc) (EC 4.1.1.4); and/or
decrease expression relative to its wild-type of at least:
secondary alcohol dehydrogenase (sAdh) (EC 1.1.1.1).

3. The method according to claim 1, wherein the off-gas from the steam methane reformer is directly brought into contact with the genetically modified homoacetogenic bacterium without an additional step of pre-treatment and/or scrubbing of the gas.

4. The method according to claim 1, wherein the homoacetogenic bacterium to be genetically modified is selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446, Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950), Butyribacterium methylotrophicum (DSM 3468), Clostridium aceticum (DSM 1496), Clostridium autoethanogenum (DSM 10061), Clostridium carboxidivorans (DSM 15243), Clostridium drakei (ATCC BAA-623), Clostridium formicoaceticum (DSM92), Clostridium glycolicum (DSM1288), Clostridium ljungdahlii (DSM 13528), Clostridium mayombei (DSM 6539), Clostridium methoxybenzovorans (DSM 12182 [SR3]), Clostridium ragsdalei (DSM 15248), Clostridium scatologenes (DSM 757), Clostridium species ATCC 29797, Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM2834), Moorella sp. HUC22-1, Moorella thermoacetica (DSM 521), Moorella thermoautotrophica (DSM 1974), Oxobacter pfennigii (DSM 3222), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030).

5. The method according to claim 1, wherein the homoacetogenic bacterium is selected from the group consisting of Clostridium autoethanogenum (DSM 10061), Clostridium ljungdahlii (DSM 13528), Clostridium carboxidivorans (DSM 15243), Acetobacterium woodii (DSM 1030), Clostridium ragsdalei (DSM 15248), Clostridium drakei (ATCC BAA-623), Moorella thermoacetica (DSM 521), Moorella thermoautotrophica (DSM 1974), Sporomusa silvacetica (DSM 10669), and Alkalibaculum bacchi (DSM 22112).

6. The method according to claim 1, wherein the gas composition further comprises N₂, O₂, and H₂S.

7. The method according to claim 1, wherein the CO₂ is in the range of 35-65% vol, the H₂ is in the range of 20-40% vol, the CO is in the range of 5-20% vol, and/or the CH₄ is in the range of 0.01-20% vol.

8. The method according to claim 6, wherein the O₂ concentration is between 0.000005%-1% volume and/or the H₂S concentration is between 0.00000001%-0.0001% volume.

9. The method according to claim 1, wherein the homacetogenic bacteria is a genetically modified Clostridium autoethanogenum or Clostridium ljungdahlii.

10. The method according to claim 1, comprising a step of
(a) receiving a stream of the off gas from the steam methane reformer.

11. An apparatus for producing acetone from a gaseous composition by microbial fermentation, the apparatus comprises:
(i) a source for the gaseous composition for continuously providing an off-gas stream from a steam methane reformer comprising at least CO, CO₂, H₂ and CH₄
(ii) an inlet on a bioreactor for receiving the off gas from the steam methane reformer,
(iii) a bioreactor comprising a culture of a genetically modified homoacetogenic bacteria, (iii) an outlet on the bioreactor for exiting of the off gas after contact with the homoacetogenic bacteria
wherein the source provides the gaseous composition coming directly from the steam methane reformer; and
the homoacetogenic bacterium is genetically modified to produce acetone from the gas composition.

12. Use of an off gas from a steam methane reformer for production of acetone, wherein the off gas comprises at least CO, CO₂, H₂ and CH₄ and the off gas is directly brought into contact with at least one genetically modified homoacetogenic bacteria, wherein the homoacetogenic bacterium is genetically modified to produce acetone from the gas composition.

13. Use according to claim 12, wherein the genetically modified homoacetogenic bacterium is selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446, Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950), Butyribacterium methylotrophicum (DSM 3468), Clostridium aceticum (DSM 1496), Clostridium autoethanogenum (DSM 10061), Clostridium carboxidivorans (DSM 15243), Clostridium drakei (ATCCBAA-623), Clostridium formicoaceticum (DSM92), Clostridium glycolicum (DSM 1288), Clostridium ljungdahlii (DSM 13528), Clostridium mayombei (DSM 6539), Clostridium methoxybenzovorans (DSM 12182 [SR3]), Clostridium ragsdalei (DSM 15248), Clostridium scatologenes (DSM 757), Clostridium species ATCC 29797, Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1, Moorella thermoacetica (DSM 521), Moorella thermoautotrophica (DSM 1974), Oxobacter pfennigii (DSM 3222), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030).

14. Use according to claim 12, wherein the off or waste gas further comprises N₂, O₂, and H₂S.

15. Use according to claim 12, wherein the off-gas from the steam methane reformer is directly brought into contact with the genetically modified homoacetogenic bacterium without an additional step of pre-treatment and/or scrubbing of the gas.


SEQUENCE LISTING (21 sequences)

SEQ ID NO: 1 - protein, 278 aa, Pantoea ananatis
MSHAYSVININKLNVFVELVKCSVPTRNRTILINGALATSSSFRNWKRFLADKSDVITFDLPFSGKSKPYNEQDCNIVTLDDEVAIIREIISIYKPNIIASASWGGASTLKLLNNGEDGLEHAIIASYSFEFNDRMRTYVKQANLYSKEKKFVDLAHLMNEEVGAYLPNKMKFCNFRHLTTLDEIEYRQGRFHLEQIANIRNHDYKSIIDNSACQFHFINGDLDIHTPQKNIEEICGESDKTCFYQIRDAGHFLDLEGKVSRERTAAAFNTIFNQIGV

SEQ ID NO: 2 - protein, 390 aa, Pantoea ananatis
MKVIIGALGSAGDVFPCIEIGSLLKEKNHDVYLLANDYFRSEAVSRGLSFVAVGDKETYVNAVQDRSLWEKKHSLKRISHYMAEQQEAMFNAMSALVDDDFIIIHSLWCFAAKAVSDKYQIKKYAISLTNSNLKLQPGRVINALEKGLRVNLNWKLALFQRALVSPALQDTLDKLRRDNHLPACRNIYADWVSGGQRSIVLYEPWFYQKKARNGFYAGFMLNKSRASTDDEQITAFIDSRTVVFFTSWALAEKKSVSQILQDLKAEGLKCVLVTPTATALSVNDDVMTVPQLNIALVETCLFAIHHGGIGTTAQLLKNGVPQLIYPRAFDQFENARALERLACGLKGKGIKEIRKMAALSRHKKHNCDYYAAQFDKAGNKGHELENFLTR

SEQ ID NO: 3 - DNA, 2011 bp, Pantoea ananatis
  source 1..2011: mol_type = genomic DNA
atgagtcacgcatactctgttataaacataaacaaacttaacgtttttgtggaattagtgaaatgcagcgtgcccacacgtaacaggaccatcctgataaatggcgcgctggcgacctcgtcttcctttcggaactggaagcggtttctggcggataaatcagatgtgattacgttcgatctgcctttttccggcaaatcgaaaccctacaatgagcaggactgtaatatcgttacgctggatgatgaagtggcgatcattcgggaaattatttcaatctataagcctaacattattgcctctgcttcgtggggcggcgcatcgacgctgaagctgttaaataacggcgaagatggcctggaacacgccattattgcgtcttactctttcgaatttaacgatcgaatgcgcacctacgttaaacaggcaaatctgtactcaaaagagaaaaaatttgtcgatcttgcgcatttgatgaacgaagaagtcggcgcctatttgccgaacaaaatgaaattctgcaatttcagacacctcaccacgctggatgaaattgagtaccgccaggggcgcttccaccttgagcaaatcgctaatattcgcaatcatgactacaaaagtattatcgataattcggcatgtcagtttcactttatcaatggcgatctggatattcatacgccgcagaaaaacattgaagagatttgcggcgagtcggacaaaacctgcttttatcagatcagagatgcgggacactttttagatctggaaggaaaagttagccgggagcgtacggctgccgcctttaacaccatttttaaccagattggtgtctgaaatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaa

SEQ ID NO: 4 - DNA, 2025 bp, synthetic construct
  source 1..2025: mol_type = other DNA
atctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaa

SEQ ID NO: 5 - DNA, 107 bp, synthetic construct
  source 1..107: mol_type = other DNA
caaataaaacgaaaggctcagtcgaaagactgggcctttcgttttatctgttgtttgtcggtgaacgctctcctgagtaggacaaatccgccgggagcggatttgaa

SEQ ID NO: 6 - DNA, 11091 bp, synthetic construct
  source 1..11091: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaatgagtcacgcatactctgttataaacataaacaaacttaacgtttttgtggaattagtgaaatgcagcgtgcccacacgtaacaggaccatcctgataaatggcgcgctggcgacctcgtcttcctttcggaactggaagcggtttctggcggataaatcagatgtgattacgttcgatctgcctttttccggcaaatcgaaaccctacaatgagcaggactgtaatatcgttacgctggatgatgaagtggcgatcattcgggaaattatttcaatctataagcctaacattattgcctctgcttcgtggggcggcgcatcgacgctgaagctgttaaataacggcgaagatggcctggaacacgccattattgcgtcttactctttcgaatttaacgatcgaatgcgcacctacgttaaacaggcaaatctgtactcaaaagagaaaaaatttgtcgatcttgcgcatttgatgaacgaagaagtcggcgcctatttgccgaacaaaatgaaattctgcaatttcagacacctcaccacgctggatgaaattgagtaccgccaggggcgcttccaccttgagcaaatcgctaatattcgcaatcatgactacaaaagtattatcgataattcggcatgtcagtttcactttatcaatggcgatctggatattcatacgccgcagaaaaacattgaagagatttgcggcgagtcggacaaaacctgcttttatcagatcagagatgcgggacactttttagatctggaaggaaaagttagccgggagcgtacggctgccgcctttaacaccatttttaaccagattggtgtctgaaatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 7 - protein, 269 aa, Pantoea sp.
MSHECSVLNINELNVYVELTKCSVPTRNRTILINGALATSSSFRNWKRFLADKSDVITFDLPFSGKSKPFNEQDCNIVTLDDEVAIIREIISIYKPNIIASASWGGASTLKLLHNGEEGLEHAIIASYSFEFNDRMRTYVKQANLYSKEKKFVDLAHLMNEEVGAYLPNKMKFCNFRHLTTLDEIEYRQGRFHLEQISNIRNHDYKRIIDNSACQFHFINGDLDIHTPQKNIEEICGESDKTRFYQIRECGTLFRPGRKGQPGANRRRL

SEQ ID NO: 8 - protein, 390 aa, Pantoea sp.
MKVIIGALGSAGDVFPCIEVGSLLKEKHHDVYLLANDYFRSEAVSRGLSFVAVGDKETYVNAVQDRSLWEKKHSLKRISYYMAEQQEAMFDAMSALVDDDCIIIHSLWCFAAKAVSDKYQIKKYAISLTNSNLKLQPGRLINALEKSFKVNLNWKLALFQRALVSPTLQDTIDKLRRDNNLPACRNIYADWVSSEQHTIVLYESWFYQKKARDGFYAGFLLNKSRASSDDDRITAFIDSRTVVFFTSWALAEKKSVAPILQDLKDEGLKCVLVTPTAKALSVKDDVMTVPQLNIALAETCLFAIHHGGIGTTAQLLKNGVPQLIYPRAFDQFENARALERLACGLKGKGIGEIRKMAALSRQKKHNCDFYAAQLDQQNNKGQELERFLTR

SEQ ID NO: 9 - DNA, 2012 bp, Pantoea sp.
  source 1..2012: mol_type = genomic DNA
atgagtcacgaatgttccgtcctaaacataaatgaacttaacgtttatgtggagttaacaaaatgcagcgttcctacgcgtaacaggaccatcctgatcaatggtgcgctggcaacgtcttcttcatttcgcaactggaaacggttcctggcagacaaatccgatgtgattaccttcgatttaccgttttctggcaaatcgaagcctttcaacgagcaggactgtaatatcgtcacgctggatgatgaagtggccatcatccgggaaattatttcaatctataagccgaacattatcgcttccgcctcatggggcggcgcatccacgctgaagctgttacataacggcgaagagggcctggaacacgccattattgcgtcttactctttcgaatttaacgatcgtatgcgcacctacgttaagcaggcaaacctgtactcgaaagagaaaaagtttgtcgatcttgcccatttgatgaatgaggaagtgggtgcgtatttgccgaataaaatgaaattctgcaacttcaggcacctgaccacgctggatgagattgagtaccggcaggggcgtttccacctggagcaaatctctaacattcgcaatcatgactacaagcgcattatcgataattcggcatgtcagtttcacttcatcaatggcgatctggatattcatactccacagaaaaacattgaagagatatgcggggaatcagataaaacacgcttttatcaaatcagagaatgcgggacactttttagacctggaaggaaaggtcagccgggagcgaaccgccgccgcctttaataccatttttagtcaaattggtgtctgaaatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaa

SEQ ID NO: 10 - DNA, 11092 bp, synthetic construct
  source 1..11092: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaatgagtcacgaatgttccgtcctaaacataaatgaacttaacgtttatgtggagttaacaaaatgcagcgttcctacgcgtaacaggaccatcctgatcaatggtgcgctggcaacgtcttcttcatttcgcaactggaaacggttcctggcagacaaatccgatgtgattaccttcgatttaccgttttctggcaaatcgaagcctttcaacgagcaggactgtaatatcgtcacgctggatgatgaagtggccatcatccgggaaattatttcaatctataagccgaacattatcgcttccgcctcatggggcggcgcatccacgctgaagctgttacataacggcgaagagggcctggaacacgccattattgcgtcttactctttcgaatttaacgatcgtatgcgcacctacgttaagcaggcaaacctgtactcgaaagagaaaaagtttgtcgatcttgcccatttgatgaatgaggaagtgggtgcgtatttgccgaataaaatgaaattctgcaacttcaggcacctgaccacgctggatgagattgagtaccggcaggggcgtttccacctggagcaaatctctaacattcgcaatcatgactacaagcgcattatcgataattcggcatgtcagtttcacttcatcaatggcgatctggatattcatactccacagaaaaacattgaagagatatgcggggaatcagataaaacacgcttttatcaaatcagagaatgcgggacactttttagacctggaaggaaaggtcagccgggagcgaaccgccgccgcctttaataccatttttagtcaaattggtgtctgaaatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 11 - protein, 278 aa, Pantoea stewartii
MSHGYSVVNINELNIYVELTKCSVPTRNRTILINGALATSSSFRNWKRFLADKSDVITFDLPFSGKSKPFNEQGCHIVTLDDEVTIIRELISIYKPNIIASASWGGASTLKLLNHGEEGLEHAIIASYSFEFNDRMRTYVKQANLFSKEKNFVELAHLMNDEVGAYLPNKMKFCNFRHLTSLDEIEYRQGRFHLEQIATLRNHDYKNIIDNSACQFHFINGDLDIHTPQKNIEEICGESDKTRFYQIRDAGHFLDLEGKRSRERTAAAFNTIFSQIGV

SEQ ID NO: 12 - protein, 390 aa, Pantoea stewartii
MKVIIGALGSAGDVFPCIEIGSLLKEKNHDVYLLANDYFRSEAVDRGLSFVAVGDKETYVKAVQDRALWDKKHALKRISHYMAEQQEAMFDAMSALVEDDAIIIHSLWCFAAKAVSEKYRLKKYAISLTNSNLKLQPGRVIHALEKSLRVSLNWKLALFQRALVSPALQRTIDTLRRDHNLPASRNIYADWVNNEPQSIVLYEPWFYQKKAREGFYAGFLLNKSRASLDDDRITDFIDSRTVVFFTSWALAEQSSVTRVLQDLKTEGLKCVLVTPTAAALAVNDDVMTLPHLNIALTETCLFAIHHGGIGTTAQLLKNGVPQLIYPCAFDQFENARALERLACGLKGKGIREIRKMAALARQKKHNCDFYAVQLANQTHRGLELERFLTC

SEQ ID NO: 13 - DNA, 2011 bp, Pantoea stewartii
  source 1..2011: mol_type = genomic DNA
atgagtcacggttattctgtagtaaacataaatgaacttaacatttatgtggagttaacaaaatgcagtgtcccgacccgtaacaggaccatcctgattaacggtgcactggcaacctcgtcttctttccgcaactggaaacgcttcctggcggataaatcagatgtcattacgtttgatcttccgttctcaggcaaatcaaaacccttcaacgaacagggctgccatatcgtcacgcttgatgatgaagtcacgattattcgcgagctgatttcaatttataagcccaatattattgcctcagcctcctggggcggcgcatcgactctcaagctcttgaatcacggtgaagagggcctggaacatgccatcatcgcgtcctattcctttgaatttaatgaccgtatgcggacttacgtgaagcaagccaatctgttttcaaaagagaagaactttgtcgagcttgcgcatttgatgaatgacgaagtgggtgcatatctgccgaataaaatgaagttctgcaacttcaggcatctgacgtcgctggatgagattgaataccgtcaggggcgtttccatcttgagcagattgccacgctgcgcaaccacgactacaaaaacattattgataactcggcctgccagtttcactttatcaacggcgacctggatattcatacgccgcaaaaaaacatcgaagagatatgcggcgagtcggataaaacgcgcttttatcaaatcagggatgcgggacattttctggatctggaaggcaagcgcagccgggaaaggaccgcagccgccttcaacactatttttagtcagattggtgtctgagatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaa

SEQ ID NO: 14 - DNA, 11092 bp, synthetic construct
  source 1..11092: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgagtcacggttattctgtagtaaacataaatgaacttaacatttatgtggagttaacaaaatgcagtgtcccgacccgtaacaggaccatcctgattaacggtgcactggcaacctcgtcttctttccgcaactggaaacgcttcctggcggataaatcagatgtcattacgtttgatcttccgttctcaggcaaatcaaaacccttcaacgaacagggctgccatatcgtcacgcttgatgatgaagtcacgattattcgcgagctgatttcaatttataagcccaatattattgcctcagcctcctggggcggcgcatcgactctcaagctcttgaatcacggtgaagagggcctggaacatgccatcatcgcgtcctattcctttgaatttaatgaccgtatgcggacttacgtgaagcaagccaatctgttttcaaaagagaagaactttgtcgagcttgcgcatttgatgaatgacgaagtgggtgcatatctgccgaataaaatgaagttctgcaacttcaggcatctgacgtcgctggatgagattgaataccgtcaggggcgtttccatcttgagcagattgccacgctgcgcaaccacgactacaaaaacattattgataactcggcctgccagtttcactttatcaacggcgacctggatattcatacgccgcaaaaaaacatcgaagagatatgcggcgagtcggataaaacgcgcttttatcaaatcagggatgcgggacattttctggatctggaaggcaagcgcagccgggaaaggaccgcagccgccttcaacactatttttagtcagattggtgtctgagatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 15 - protein, 295 aa, Pseudomonas aeruginosa
MRRESLLVSVCKGLRVHVERVGQDPGRSTVMLVNGAMATTASFARTCKCLAEHFNVVLFDLPFAGQSRQHNPQRGLITKDDEVEILLALIERFEVNHLVSASWGGISTLLALSRNPRGIRSSVVMAFAPGLNQAMLDYVGRAQALIELDDKSAIGHLLNETVGKYLPPRLKASNHQHMASLATGEYEQARFHIDQVLALNDRGYLACLERIQSHVHFINGSWDEYTTAEDARQFRDYLPHCSFSRVEGTGHFLDLESKLAAVRVHRALLEHLLKQPEPQRAERAAGFHEMAIGYA

SEQ ID NO: 16 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaa

SEQ ID NO: 17 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 18 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaa

SEQ ID NO: 19 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 20 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaa

SEQ ID NO: 21 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca